(19) United States
(12) Patent Application Publication
(10) Pub. No.: US 2010/0093070 A1
(43) Pub. Date: Apr. 15, 2010

(54) TEST TUBE FOR PERFORMING SAFE AND ACCURATE URINE ANALYSES IN PATIENTS BEING SUBJECTED TO AN ANTIBIOTIC THERAPY

(76) Inventor: Renato Coco, CATANIA (IT)

Correspondence Address: HEDMAN & COSTIGAN P.C., 1185 AVENUE OF THE AMERICAS, NEW YORK, NY, 10036, US

(21) Appl. No.: 12/410,104

(22) Filed: Mar. 24, 2009

(30) Foreign Application Priority Data
Oct. 9, 2008 (IT) MI2008A001784

(51) Int. Cl. C12M 1/00

(52) U.S. Cl. 435/288.1

(57) ABSTRACT

An improved test tube for performing safe and accurate urine analyses in patients being subjected to an antibiotic therapy, comprises a container body, made of a plastics material and including a threaded plug, the container body being lined by an inhibiting substance which is layered on the wall and bottom of the test tube.

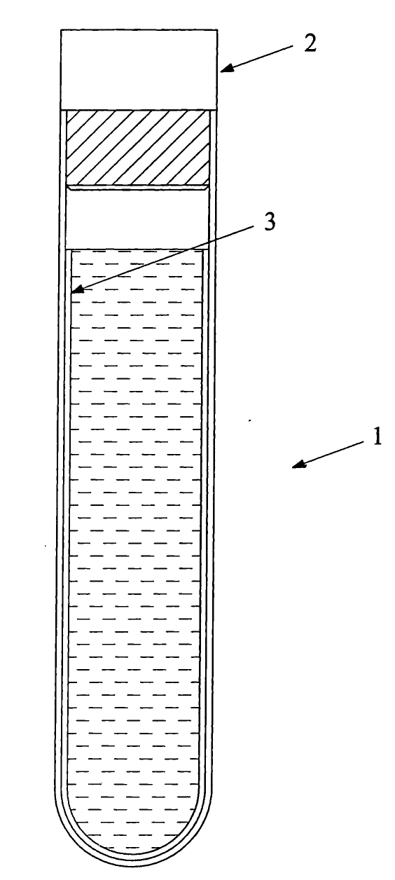

### BACKGROUND OF THE INVENTION

The present invention relates to an improved test tube for performing safe and accurate urine analyses in patients being subjected to an antibiotic therapy.

As is known, an accurate bacteriologic urine test, for searching common germs, not only must accurately establish a possible presence of bacteria in the urine sample, but also provide the concentration thereof.

At present, only bacteriuria larger than 10⁵ CFU (colony forming units)/ml are considered as significant for a diagnosis, whereas small bacteriura, less than 10⁴ CFU/ml, are generally attributed to contaminating processes.

Middle value bacteriuria, on the other hand, are considered as suspicious, that is to be verified by repeating the test and to be interpreted while taking in consideration some elements, such as clinic symptomatology, a possible antibiotic treatment, the collecting and sample transport procedure.

A very important problem to be considered is that, frequently, a patient is subjected to an antibiotic therapy, which is one of the most important causes of false analysis negative results, since antibiotic products present in urines provide an inhibiting effect to the germs, with consequent erroneous analysis negative responses.

### SUMMARY OF THE INVENTION

Accordingly, the aim of the present invention is to provide such an improved test tube construction allowing to further improve the urine analysis procedure.

Within the scope of the above mentioned aim, a main object of the invention is to provide such a test tube construction allowing to greatly improve the analysis analytic response both from a quality and an analysis facility and time reduction standpoint.

Another object of the present invention is to provide such a test tube construction which, owing to its specifically designed features, is very reliable and safe in operation.

Yet another object of the present invention is to provide such a test tube construction which can be easily made and which, moreover, is very competitive from a mere economic standpoint.

According to one aspect of the present invention, the above mentioned aim and objects, as well as yet other objects, which will become more apparent hereinafter, are achieved by an improved test tube construction adapted to prevent erroneous diagnoses in urine analysis procedures on patients being subjected to an antibiotic therapy, characterized in that said test tube construction comprises a container body made of a plastics material and including a threaded plug, said container body being lined by an inhibiting substance which is layered on a wall and bottom of said test tube thereby, as urine containing antibiotic substances is filled in said test tube construction, said antibiotic substances are segregated by said inhibiting substance to prevent said antibiotic substances from inhibiting a bacteria growth.

### BRIEF DESCRIPTION OF THE DRAWING

Further characteristics and advantages of the present invention will become more apparent hereinafter from the following detailed disclosure of a preferred, though not exclusive, embodiment of the invention, which is illustrated, by way of an indicative, but not limitative example, in the accompanying drawing, the sole FIGURE of which shows a longitudinal cross sectional view of a test tube construction according to the present invention.

### DESCRIPTION OF THE PREFERRED EMBODIMENTS

With reference to the number references of the above mentioned figure, the improved test tube construction according to the invention, which has been generally indicated by the reference number 1, comprises a pierceable plug element 2, and a film 3 of an inhibiting substance arranged in layers on the wall and bottom of the test tube.

According to the invention, the improved test tube is a vacuum test tube 1 made of a plastics material, preferably a PET material, and the plug element 2 is a threaded plug element.

Thus, the use of PET, instead of a glass material, is such as to increase the operating safety, whereas the threaded coupling system will provide plug handling characteristics better than those of a pressure applied plug.

In fact, with urine being sucked through the plug central rubber element, the urine test is facilitated since the test tube can be easily screwed off and then also easily relocated.

According to the invention, the layered inhibiting substance, comprising active resins or carbon materials, is coated on the bottom of the test tube, and also arranged in layers on the walls of the latter, by a coating process consisting of depositing, by dipping, thin films of a solution (active carbons) on a solid substrate (the wall of the test tube), removing any excess materials and then drying the thus made test tube, thereby providing great advantages including a larger resin and urine contact surface, allowing to increase the testing method sensitivity, (owing to a larger contact between the resin and antibiotic materials), and greatly reduce the urine testing reaction time.

In fact, the mentioned high contact surface deriving from a layering of the resin materials on the inner wall of the test tube, allows said resin material to affect a larger urine volume, thereby providing a very great inhibiting effect, while in turn greatly improving the test sensitivity, which feature is very important for urine sample contaminated by very small antibiotic volumes.

According to the present invention, it is further preferred to use, instead of said resin materials, active carbons having a larger polluting substance absorbing and removing efficiency.

In fact, as is known, one of the main features of active carbons is their porosity, that is:

pores with a diameter less than 2 nm (micropores)

pores with a diameter from 2 to 50 nm (mesopores), and

pores with a diameter larger than 50 nm (macropores).

The active carbon absorption properties being generally characterized by the following features:

an adsorption capability owing to which the solute adheres to the solid surface;

an absorption capability causing the solute to be diffused through a porous solid material thereby adhering to the inner surfaces; and

a chemioabsorption capability, owing to which the solute is absorbed by a chemical reaction.

To the above it is to be added that two active carbon types are at present commercially available, that is:

GAC—a granular active carbon, to be used when a large inner area and small pores are desired.

PAC—an active carbon comprising smaller particles to be used when a small inner area and large pores are desired.

Active carbons are surprisingly efficient also in removing anions and cations present in liquids, in particular antibiotic substances contaminating urine.

Thus, the increase of the contact surface between urine and coated test tube (both by resins and active carbons) greatly improves the test procedure sensitivity, reduces the analysis time and increases the inhibiting capability even at low antibiotic amounts.

Moreover, the use of a plastics material (PET) test tube makes the operator works easier, while said threaded plug provides a much more flexible operation capability, in particular in manual test operations.

Thus, the inventive test tube will safely prevent erroneous diagnoses in urine analyses on patients under antibiotic therapy, since said test tube contains therein antibiotic substances absorbing resins, comprising polydivynilbenzene preferably in a micro-ball form, with a diameter from 80 to 100 Amstrong's, thereby providing a very great absorption capability, due to their specifically designed structural factors, such as surface, pore size and polarity.

Advantageously, said resins comprise two resin types, including either clear water repellent resin granules, attracting the hydrophobic part of the antibiotic substances, or dark granule resins, having such a hydrophilic nature to attract the antibiotic positively charged cationic part.

Thus, strong chemical bindings formed between the resin and drug will prevent the antibiotic substance from inhibiting any bacteric growths.

In fact, on each granule thousands binding sites for binding the antibiotic substances thereon are formed.

The granules can have a diameter of 100-200 times the bacteria diameter, thereby providing a bacteria growth facilitating surface.

It is also possible to provide a mechanical stirring for lysing away white cells and releasing intracellular organisms, under a mechanical type of action causing a breakage of bacteria aggregates, which mechanism will increase positive results of the analyzed sampled, while reducing false negative results causing an increase of hospital recovery time and negatively affecting a quick and accurate test diagnosis.

From a statistic standpoint, the repetition of the culture test on each non suitable sample is of about 10% for hospitalized patients and increases to 30-40% for non hospitalized ones.

Actually, a main cause of such an analysis repetition is due to a poor therapeutic information to the patient, thereby the therapy would be suspended at least three day before taking the urine sample, to prevent antibiotic residue substances from contaminating urine.

Advantageously, optimum results are herein achieved by using the inventive vacuum test tube 1, comprising a pierceable plug 2, assuring a vacuum condition, and already containing therein the above disclosed resin material, as schematically indicated by the reference number 3.

Alternately, as stated, instead of using said resin it is also possible to use active carbons.

The test tube, with urine filled therein, must be stirred for at least two hours, to allow the resin material to provide its antibiotic substance inhibiting activity.

In such a procedure, the test tube can also be easily used on automatic instruments, directly taking the urine sample by perforating the plug, or by manually opening the test tube and using urine held therein, thereby greatly reducing the need of repeating the test, and all the costs related to a hospitalization of the patients, while also greatly reducing non targeted antibiotic therapy substances.

Another important advantage of the invention is that the inventive test tube also contributes to greatly reducing sanitary expenses related to urinary pathologies and related infective complications.

It has been found that the invention fully achieves the intended aim and objects.

In practicing the invention, the used materials, as well as the contingent size and shapes, can be any, depending on requirements.

1. An improved test tube construction for preventing erroneous diagnoses in urine analysis procedures on patients being subjected to antibiotic therapies, characterized in that said test tube construction comprises a container body, made of a plastics material and including a threaded plug, said container body being lined by an inhibiting substance which is layered on a wall and bottom of said test tube.

2. An improved test tube construction, according to claim 1, characterized in that as an antibiotic substance containing urine is introduced into said test tube construction, an antibiotic substance in said urine is segregated by said inhibiting substance thereby preventing said antibiotic substances from inhibiting bacterial growth.

3. An improved test tube construction, according to claim 1, characterized in that said layered inhibiting substance comprises either resins or active carbons, and being coated on the bottom of said test tube and also layered on the wall of said test tube.

4. An improved test tube construction, according to claim 1, characterized in that said inhibiting substance is coated on said bottom and wall of said test tube by coating process comprising depositing by dipping thin films of an active carbon based solution on a solid substrate forming said wall of said test tube, successively removing any excess material and drying the thus coated test tube.

5. An improved test tube construction, according to claim 1, characterized in that said test tube construction comprises a pierceable plug element.

6. An improved test tube construction, according to claim 1, characterized in that said test tube is a vacuum test tube made of a plastics material, preferably PET, said plug element being a screw on and off plug element.

7. An improved test tube construction, according to claim 1, characterized in that said resin material comprises polydivynilbenzene.

8. An improved test tube construction, according to claim 1, characterized in that said resin material is in a form of microball granules.

9. An improved test tube construction, according to claim 1, characterized in that said granulated resin material comprises hydrophobic clear granules adapted to attract an hydrophobic part of said antibiotic substances.

10. An improved test tube construction, according to claim 1, characterized in that said resin material comprises dark hydrophilic granules adapted to attract a positively charged cationic part of said antibiotic substance.

11. An improved test tube construction, according to claim 1, characterized in that said test tube construction is adapted to have urine stirred therein for a stirring period of at least two hours.

12. An improved test tube construction, according to claim 1, characterized in that said test tube construction comprises a vacuum containing element with a pierceable plug element adapted to hold in said test tube a vacuum condition.

13. An improved test tube construction, according to claim 1, characterized in that said test tube construction contains therein an antibiotic substance absorption resin material.
